(19) United States
(12) Patent Application Publication
(10) Pub. No.: US 2012/0251622 A1
(43) Pub. Date: Oct. 4, 2012

(54) CELLULOSE GUM AND POLYOL TROCHE

(76) Inventor: Jeffrey T. Haley, Mercer Island, WA (US)

(21) Appl. No.: 13/478,041

(22) Filed: May 22, 2012

Related U.S. Application Data
(63) Continuation-in-part of application No. 11/800,381, filed on May 4, 2007.

(51) Int. Cl. A61K 9/20; A61K 47/38

(52) U.S. Cl. 424/465; 514/781

(57) ABSTRACT

A troche with 2.5-6% cellulose gum and at least 75% polyol molecules is disclosed. It may be an adherent troche, preferably adhered to teeth or gums or cheek with acacia gum. Consumers are instructed to use the troches to relieve the sensations of dry mouth, particularly while sleeping. The polyol molecules are preferably xylitol. The troches may be made with a bi-layer tablet press rounded on one side and flat on the other, preferably using acacia gum in the flat side layer for adhesion.

This application is a continuation in part of U.S. patent application Ser. No. 11/800,381 filed May 4, 2007 and claims priority from U.S. provisional patent application 60/879,846 filed Jan. 11, 2007.

### BACKGROUND

Cellulose gum, consisting of one or more of carboxymethylcellulose, methylcellulose, and hydroxypropylmethylcellulose, when dissolved in saliva in the mouth, can be an effective oral lubricant and humectant to relieve the sensations of dry mouth. However, in excessive concentration, cellulose gum has an unattractive mouth feel of being tacky or gloopy, and merely dissolving and diluting with saliva will not solve this problem. To achieve an acceptable mouth feel from cellulose gum placed dry into the mouth, the cellulose gum must be diluted with an ingredient other than water to create a combination that is an effective oral lubricant and humectant.

Oral care researchers have established that frequent delivery of xylitol molecules in the mouth can reduce caries, gingivitis, periodontitis, halitosis and inner ear infections by suppressing the growth of certain bacteria. These bacteria thrive on certain carbohydrate molecules such as sucrose, glucose, fructose and other sugars but, when they ingest the xylitol molecules, they cease proliferating and cease to adhere to human tissues. Delivering xylitol molecules in the mouth also provides other benefits, such as remineralization of teeth and reduction of plaque and halitosis by stimulating saliva flow.

Mints, lozenges, and lollipops may be technically described as “troches”. For treatment of health problems in the mouth or throat, people have for centuries held in their mouths a composition containing medication for topical application. Since the middle ages, the name for such a composition, derived from Latin and previously from Greek, is “troche”. A modern form of troche is the cough drop, so named because it was formed by “dropping” hot, viscous, sugar-based candy onto a sheet or into a mold where it cools to form the troche. Another modern form of troche is the “lozenge”, so named because it was in the shape of a diamond (like on playing cards), which is the meaning of the word “lozenge”. A troche is large enough that a person is able to track where it is in the mouth and move it with their tongue, that is, larger than about 5 mm in at least two dimensions.

U.S. Pat. No. 6,139,861 issued to Mark Friedman surveys methods for adhering a troche to a location within the mouth. These methods include two forms of adherent troches, referred to by Friedman as a “mucoadhesive erodible tablet”. These tablets are formed using adhesive polymers such as polyacrylic acid, and carbopol-934.

### SUMMARY OF THE INVENTION

The inventor has discovered, surprisingly, that, in a troche, polyol molecules of at least 75% can effectively dilute cellulose gum molecules within a range of 2.5% -6% cellulose gum in the troche to achieve an attractive mouth feel, and the combination is an effective, synergistic oral lubricant and humectant.

In one aspect, the invention is a composition of 2.5% -6% cellulose gum diluted with at least 75% solid phase polyol molecules, particularly xylitol, and essentially nothing else but perhaps a small amount of flavor, tablet compression binder, and mechanical lubricant for the process of tablet pressing. The composition dissolves much more slowly than substantially pure (greater than or equal to 98%) xylitol or xylitol with flavors added. The composition is comprised of crystallized polyol molecules intermixed with molecules of cellulose gums that swell when exposed to water. By their binding to water molecules and swelling, the gum molecules block the flow of water to the polyol molecules and slow dissolution. The cellulose gum molecules may be one or more of carboxymethylcellulose, methylcellulose, and hydroxypropylmethylcellulose. Instead of xylitol, the polyol molecules may be any polyol that is a solid at room temperatures, such as erythritol, sorbitol, mannitol, maltitol, isomalt, and lactitol.

The composition may be formed by pressing powders of polyol crystals and one or more cellulose gums into a tablet with a tablet press such that the composition will have polyol crystals as large as grains of powder and the molecules of cellulose gums are one or more of intermixed within the grains or a coating on the grains or clumped into their own grains as large as grains of powder. The composition may be formed into a troche. In an embodiment, the dissolution time of the troche in a human mouth is, on average, more than 25 minutes.

In another aspect, the invention is an adherent troche, at least 5 mm in each of at least two dimensions, that, when held in a human mouth, remains in the mouth as a single item that will not spread to be in a plurality of locations in the mouth at one time and erodes, thereby releasing cellulose gum and polyol molecules over time. The troche may be a rigid, pressed powder, adherent tablet. In an embodiment, the polyol molecules are xylitol. Alternatively, the polyol molecules may comprise one or more of erythritol, sorbitol, mannitol, maltitol, isomalt, and lactitol.

The adhesive molecules may comprise acacia gum. Alternatively, they may comprise one or more of gelatin, alginate, starch, pectin, polyvinylpyrolidone, carboxymethylcellulose, hydroxymethylcellulose, polyvinyl acid, polyacrylic acid, and carbopol.

The troche may comprise two layers, a first layer comprised of, by dry weight, 2.5% to 6% cellulose gums molecules and at least 75% solid phase polyol molecules and a second layer comprised of, by dry weight, at least 30% adhesive molecules, which may be acacia gum (aka gum arabic). The troche has a planar shape with a width greater than 5 mm and a thickness less than the width. In an embodiment, one side of the thickness comprises, by dry weight, at least 50% acacia gum. The acacia gum adheres very well to teeth and gums.

The portion of the troche that includes the side with acacia gum may be formed by pressing powders into a tablet with a tablet press. The rest of the troche may be formed by adhering another layer to the acacia gum layer. In a preferred embodiment, the troche is a bi-layer tablet and a first layer, which includes the first side, comprises at least 80% acacia gum. Alternatively, the adhesive side may be formed by mixing the adhesive into a paste with a solvent, forming a blob with the paste, and then removing most of the solvent to form a planar shape. The solvent may be water.

The oral adhesive tablet may be made by configuring a bi-layer tablet press having a die and lower and upper punches such that the lower punch is dish shaped to produce a rounded tablet surface and the upper punch is substantially flat. One makes tablets with the press by first pouring into the die a granular material that is not intended to be oral adhesive, then tamping the granular material with the upper punch, then adding to the die oral adhesive granular material, then compressing the granular materials between the two punches to form a tablet that is substantially flat on an oral adhesive side and rounded on the other side.

The dish shape may be approximately a portion of a sphere. The dish shape may be produced by a face on a lower punch that is substantially flat in a center area and the center area is surrounded by a raised edge which forms a dish shape.

In another aspect, the invention is a method for combating the sensation of dry mouth by providing dissolving troches comprising 2.5-6% cellulose gum and at least 75% crystalline xylitol which, when exposed to saliva in a human mouth, on average, release cellulose gum and xylitol molecules more slowly than a troche of substantially pure xylitol (greater than 98% purity) and instructing consumers of the troches to place a troche in a mouth and keep it there until the cellulose gum and xylitol in the troche are dissolved. In one embodiment, the dissolution time of the troches in a human mouth is, on average, more than 25 minutes at typical mid day levels of saliva flow. The troches may have a mucoadhesive layer on one side for adhering to teeth and/or gums.

### BRIEF DESCRIPTION OF THE DRAWING

FIG. 1 shows a side view or cross section of a bi-layer adherent troche made with a tablet press.

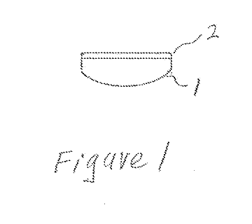

### DETAILED DESCRIPTION

The composition of cellulose gum and solid phase polyol molecules, particularly xylitol, is made by mixing polyol molecules with substantial amounts of cellulose gum, including carboxymethylcellulose (CMC), methylcellulose, and hydroxypropylmethylcellulose. Instead of xylitol, the polyol molecules may be any polyol that is solid at room temperatures, such as erythritol, sorbitol, mannitol, maltitol, isomalt, and lactitol.

High viscosity carboxymethylcellulose (CMC 15000 from TIC Gums) required only 2.4% to be effective. A preferred embodiment has 3.4-5% low viscosity carboxymethylcellulose (CMC 15 from TIC Gums) and 92-96.6% xylitol with small amounts of mint oil for flavor, magnesium stearate for lubricant with contacted machine surfaces, and tablet compression binder.

The composition may be formed by pressing powders of polyol crystals and one or more gums into a tablet with a tablet press. Xylitol grains of 50 to 350 microns are preferred. The grains may be granulated with a coating of cellulose gum on the outside, such as Danisco Xylitab 200 which is granulated with up to but less than 2% carboxymethylcellulose (CMC). This is not enough CMC to achieve the desired lubrication of a dry mouth. Adding at least 1.2% powdered CMC 15 from TIC Gums is effective. Adding 2.1% to 3.5% is preferred, depending on how much CMC is on the xylitol grains and the viscosities of both the CMC on the grains and the added powdered CMC. A 0.7 gram troche about 4.5 mm thick dissolved in 47 minutes in the mouth with 1.2% added CMC 15. With 2.5% added CMC 15, the dissolution rate was 90 minutes. With 3.5% added CMC 15, the dissolution rate was 120 minutes. A 0.5 gram troche with 3.4% low viscosity CMC dissolved in 40-120 minutes, depending on saliva flow.

Alternatively, grains of pure xylitol, such as Danisco Xylitab 300, may be mixed with about 5% cellulose gum powder and then pressed.

Alternatively, an embodiment may be made by coating fine grains of xylitol with 2.5% -6% carboxymethylcellulose (CMC) (by weight relative to the xylitol) and incorporating these grains into a tablet.

The embodiment of an adherent troche that, when held in a human mouth, erodes, thereby releasing cellulose gum and polyol molecules over time, allows delivery of polyol molecules without the effect on appearance of chewing or having a mint in one's mouth. It can also be used while sleeping which is when the problems of dry mouth are worst.

In preferred embodiments of the oral adhering disc, the troche comprises, by dry weight between 50% and 90% solid phase polyol molecules, particularly xylitol. Greater amounts are unachievable because at least 10% is needed for the adhesive and cellulose gum. This leaves between 10% and 50% for the adhesive molecules that adhere in a human mouth as well as cellulose gum molecules.

The adhesive molecules may comprise acacia gum. Acacia gum adheres very well to teeth and gingiva, which are the preferred locations for adhesion, and it does not dissolve too fast or leave an unattractive mouth feel. On the surface designed to be adherent, between 80% and 100% acacia gum is preferred for good adhesion. Alternatively, the adhesive molecules may comprise one or more of gelatin, alginate, starch, pectin, polyvinylpyrolidone, carboxymethylcellulose, hydroxymethylcellulose, polyvinyl acid, polyacrylic acid, and carbopol.

The adherent layer 2 can be quite thin. In tests on a preferred size of troche, about 11.5 mm in diameter by 4 to 5 mm thick, the preferred thickness of a layer of about 99% acacia gum was about one-half millimeter. This can be made by bi-layer tablet pressing or by depositing a paste of acacia gum into a mold or by extrusion and die cutting.

The troche may comprise two layers, a first layer 1 comprised of, by dry weight, at least 75% solid phase polyol molecules and a second layer 2 comprised of, by dry weight, at least 30% adhesive molecules. To minimize adhesive gums required and minimize size for the amount of cellulose gum and polyol delivered, making a bi-layer troche is preferred.

The preferred embodiment of the troche is made on a bi-layer tablet press, putting 85 to 95% of the total weight into a polyol layer 1 of about 90 to 97% polyol and 5 to 15% of the weight into an adhesive layer 2 of 30 to 99% adhesive gums. A pressed powder bi-layer xylitol troche, 12 mm in diameter and 4 to 5 mm thick with one-half millimeter of 99% acacia gum in one layer and 5% CMC cellulose gum in the xylitol as described above, with small amounts of flavor (up to 4%), tablet compression binder such as Klucel by Aqualon (up to 2%), and magnesium stearate mechanical lubricant (or calcium stearate or stearic acid) (up to 1%) (total up to 7% being neither polyol nor cellulose gum), adheres well and dissolves in about 40-90 minutes.

When making bi-layer tablets with a typical press, a first powder is placed in the die, sitting on the lower punch, then the upper punch tamps the powders, leaving the surface having the shape of the upper punch face, then powders of the second layer are added, then an upper punch presses again.

A method for making a rounded bi-layer oral adhesive tablet on a typical bi-layer press is to configure the press to have a lower punch that is dish shaped to produce a rounded tablet surface and an upper punch that is substantially flat. One makes tablets with the press by first pouring into the die a granular material that is not intended to be oral adhesive, then tamping the granular material with the upper punch, then adding to the die oral adhesive granular material, then compressing the granular materials between the two punches to form a tablet that is substantially flat on an oral adhesive side and rounded on the other side.

The dish shape may be approximately a portion of a sphere. The dish shape may be produced by a face on a lower punch that is substantially flat in a center area and the center area is surrounded by a raised edge which forms a dish shape. For a troche 12 mm in diameter, a suitable amount of dish is 1.5 to 3 mm, preferably 2.1 mm, with a total tablet thickness of 4 to 5 mm.

The troches described above may be used for relieving the sensation of dry mouth. Dissolving troches comprising cellulose gum and crystalline xylitol which, when exposed to saliva in a human mouth, on average, release cellulose gum and xylitol molecules more slowly than a troche of pure xylitol are supplied to consumers. The consumers are instructed to place a troche in their mouths and keep it there until the troche is dissolved. The greater the number of hours each day with a troche releasing cellulose gum and xylitol in the mouth, the better, up to a point of diminishing returns. Using two troches as described above at the end of each day, while sleeping, and one after each meal, is presently preferred.

Users adhere a troche to a tooth or adjoining gums in the rear of their mouths at any time of day or night, preferably after each meal or snack, at least four times per day. Placing it on the tongue side of the teeth causes it to erode more quickly than placing it on the cheek side. It can instead be adhered to the cheek.

While particular embodiments of the invention have been described above the scope of the invention should not be limited by the above descriptions but rather limited only by the following claims.

1. A tablet, comprising:
compressed, blended powders, the powders consisting of
(a) between 2.5% and 6% by weight cellulose gum molecules,
(b) with or without flavor molecules, mechanical lubricant molecules for contact with machine surfaces, tablet compression binder molecules, and impurities, and
(c) at least 75% by weight polyol molecules making up the balance.

2. The tablet of claim 1 wherein the polyol molecules are at least 90% by weight.

3. The tablet of claim 1 wherein the flavor molecules, mechanical lubricant molecules for contact with machine surfaces, compression binder molecules, and impurities comprise no more than 7% by weight.

4. The tablet of claim 1 further comprising:
adhered by compression to one side of the tablet, a mucoadherent, compressed powder layer, the tablet being a bi-layer tablet.

5. The tablet of claim 1 wherein the cellulose gum molecules are selected from the group consisting of carboxymethylcellulose, methylcellulose, and hydroxypropylmethylcellulose.

6. The tablet of claim 1 wherein the polyol molecules are selected from the group consisting of xylitol, erythritol, sorbitol, mannitol, maltitol, isomalt, and lactitol.

7. The tablet of claim 1 with flavor molecules consisting of mint oil.

8. The tablet of claim 1 with mechanical lubricant molecules consisting of stearate selected from the group consisting of magnesium stearate, calcium stearate, and stearic acid.

9. A method for relieving the sensations of dry mouth comprising:
holding in the mouth a tablet of compressed, blended powders, the powders consisting of
(a) between 2.5% and 6% by weight cellulose gum molecules,
(b) with or without flavor molecules, tablet compression binder molecules, mechanical lubricant molecules for contact with machine surfaces, and impurities, and
(c) at least 75% by weight polyol molecules making up the balance.

10. The method of claim 9 wherein the polyol molecules are at least 90% by weight.

11. The method of claim 9 wherein the flavor molecules, mechanical lubricant molecules for contact with machine surfaces, compression binder molecules, and impurities comprise no more than 7% by weight.

12. The method of claim 9 wherein the cellulose gum molecules are selected from the group consisting of carboxymethylcellulose, methylcellulose, and hydroxypropylmethylcellulose.

13. The method of claim 9 wherein the polyol molecules are selected from the group consisting of xylitol, erythritol, sorbitol, mannitol, maltitol, isomalt, and lactitol.

14. The method of claim 9 wherein the tablet further comprises:
adhered by compression to one side of the tablet, a mucoadherent, compressed powder layer, the tablet being a bi-layer tablet.

15. The method of claim 9 with flavor molecules consisting of mint oil.

16. The method of claim 9 with mechanical lubricant molecules consisting of stearate selected from the group consisting of magnesium stearate, calcium stearate, and stearic acid.
